(19) United States
(12) Patent Application Publication
(10) Pub. No.: US 2024/0335629 A1
(43) Pub. Date: Oct. 10, 2024

(54) AUTOMATIC SHUTOFF OF GAS SUPPLY TO PATIENT WORN DEVICE

(71) Applicant: BeaconMedaes LLC, Rock Hill, SC (US)

(72) Inventors: Jay D’Agostino, Coram, NY (US); Greg DelBene, Davidson, NC (US)

(21) Appl. No.: 18/472,838

(22) Filed: Sep. 22, 2023

Related U.S. Application Data
(60) Provisional application No. 63/494,072, filed on Apr. 4, 2023.

(51) Int. Cl. A61M 16/00; A61M 1/00

(52) U.S. Cl. CPC A61M 16/022; A61M 1/74; A61M 1/772; A61M 2016/0027; A61M 2202/0208; A61M 2205/3358

(57) ABSTRACT

A method for automatic shutoff of a gas supplied from a SOT device to a patient worn device, including controlling a flow of gas to a patient worn device at a pressure greater or less than atmospheric pressure, detecting a pressure drop to atmospheric pressure, and in response to the detecting, automatically discontinuing the flow of the gas to the patient worn device. A pod, a pod system, and an accessory insertable within the pod are also provided.

### CROSS-REFERENCE TO RELATED APPLICATIONS

This application claims the benefit of and priority to U.S. Provisional Application No. 63/494,072, filed Apr. 4, 2023, and entitled “Automatic Shutoff of Gas Supply to Patient Worn Device.”

### FIELD OF TECHNOLOGY

The following relates to embodiments for controlling flow of gas to a patient worn device, and more specifically to embodiments of a pod accessory having a sensor to detect pressure drops, a pod system, and a method thereof.

### BACKGROUND

Conventional Suction and Oxygen Therapy (SOT) in medical facilities include flow regulators for medical gas and suction regulators that are connected to existing ports located on a wall of a room in a medical facility. Tubes connect to the flow regulators and suction regulators and then connect to the patient or medical equipment used to treat a patient. The connection to the medical equipment worn by the patient and the flow regulator can be interrupted, which can result in unwanted flow of gas into an environment.

### SUMMARY

An aspect relates to a method comprising controlling a flow of gas to a patient worn device at a pressure greater or less than atmospheric pressure, detecting a pressure drop to atmospheric pressure, and in response to the detecting, automatically discontinuing the flow of the gas to the patient worn device.

In an exemplary embodiment, a flow management device of a suction and oxygen therapy device controls the flow to the patient worn device. A fluidic connection is established by tubing connected at a first end to the flow management device and at a second end to the patient worn device.

The pressure drop is caused by a disconnection of the fluidic connection between the flow management device and the patient worn device. The disconnection is a result of the first end of the tubing being removed from an outlet of the flow management device. In some instances, the disconnection is a result of the second end of the tubing being removed from the patient worn device. In response to detecting the pressure drop, a valve is closed to automatically discontinue the flow of the gas to the patient.

Another aspect relates to a method comprising regulating, by a flow management device of a pod, a flow of gas to a patient worn device at a pressure greater or less than atmospheric pressure, the pod configured to receive an accessory having an outlet and a pressure sensor disposed proximate the outlet of the accessory, receiving, by a control interface of the pod, a signal from the pressure sensor of the accessory indicating that the pressure has dropped to atmospheric pressure, and transmitting, by the control interface of the pod, a control signal to the flow management device to stop the flow of gas to the patient worn device.

In an exemplary embodiment, a valve of the flow management device is closed as a function of the control signal. The control signal is sent when the pressure drop detected is caused by a disconnection of a fluidic connection between the flow management device and the patient worn device; the fluidic connection is established by tubing connected at a first end to the outlet of the accessory and at a second end to the patient worn device.

Another aspect relates to a removable accessory for use with a pod, the removable accessory comprising: a body portion having a first side and a second side, at least one fluidic coupling disposed on the body portion configured to be fluidically coupled to a flow management device of the pod, as a function of the removable accessory being inserted within a pod casing of the pod, an outlet fluidically connected to the at least one fluidic coupling, disposed on the body portion and configured to be attached to a tubing that fluidically connects the removable accessory to a patient worn device, and a sensor disposed proximate the outlet and configured to detect a pressure differential within the body portion of the accessory.

In an exemplary embodiment, the sensor transmits a signal to the pod to discontinue a flow of gas to the patient worn device if the pressure differential indicates a pressure proximate the outlet is at or near atmospheric pressure.

In an exemplary embodiment, the sensor is integrated within the body portion, and in fluidic communication with the tubing attached to the outlet.

Another aspect relates to a system comprising: a pod comprising a flow management device disposed within a pod casing, at least one fluidic coupling within the casing that is fluidically connected to the flow management device, a removable accessory for use with the pod, the removable accessory comprising: a body portion having a first side and a second side, at least one fluidic coupling disposed on the body portion that fluidically connects with the at least one fluidic coupling within the casing of the pod as a function of the removable accessory being inserted into the pod, and an outlet, a sensor configured to detect a change in pressure at the outlet of the removable accessory, and a control interface electrically connected to the sensor and configured to receive a signal from the sensor indicating the change in pressure, wherein the control interface sends a control signal to the flow management device to stop a gas from flowing through the outlet to a patient worn device.

In an exemplary embodiment, the system further includes a tubing connected at a first end to the outlet of the removable accessory and at a second end to the patient worn device, so that the gas delivered to the patient worn device flows from the outlet to the patient worn device when the tubing is connected at both the first end and the second end of the tubing. The change in pressure reflects a pressure drop to atmospheric pressure at the outlet as a result of the patient worn device being fluidically disconnected from the outlet. For instance, the change in pressure reflects a pressure drop to atmospheric pressure at the outlet as a result of the tubing being removed from the outlet.

Another aspect relates to a pod comprising: a flow management device disposed within a pod casing, the flow management device electrically coupled to a control interface, at least one fluidic coupling within the casing that is fluidically connected to the flow management device, the at least one fluidic coupling configured to fluidically connect with a removable accessory insertable within the pod casing, and a receptacle within the casing that is configured mate with a pressure sensor of the removable accessory, the receptacle electrically coupled to the control interface so that when the removable accessory is inserted within the pod casing, data from the pressure sensor is received by the control interface.

The foregoing and other features of construction and operation will be more readily understood and fully appreciated from the following detailed disclosure, taken in conjunction with accompanying drawings.

### BRIEF DESCRIPTION OF THE DRAWINGS

Some of the embodiments will be described in detail, with reference to the following figures, wherein like designations denote like members, wherein:

FIG. 1 depicts a flowchart of a method for automatically discontinuing a flow of gas, in accordance with embodiments of the present invention;

FIG. 2A depicts a schematic view of an SOT device connected to a patient worn device via tubing, in accordance with embodiments of the present invention;

FIG. 2B depicts a schematic view of the SOT device disconnected from the patient worn device, in accordance with embodiments of the present invention;

FIG. 3 depicts a schematic view of a rough-in assembly having connections to a plurality of sources, in accordance with embodiments of the present invention;

FIG. 4 depicts a schematic view of pods insertable into the rough-in assembly of FIG. 3, in accordance with embodiments of the present invention;

FIG. 5A depicts a schematic view of a pod inserted into the rough-in assembly to allow access to a first source;

FIG. 5B depicts a schematic view of a pod inserted into the rough-in assembly to allow access to a second source;

FIG. 5C depicts a schematic view of a pod inserted into the rough-in assembly to allow access to a third source;

FIG. 6 depicts a schematic view of a pod inserted within the rough-in assembly, in accordance with embodiments of the present invention;

FIG. 7A depicts a schematic view of a backside of a first pod, in accordance with embodiments of the present invention;

FIG. 7B depicts a schematic view of a backside of a second pod, in accordance with embodiments of the present invention;

FIG. 7C depicts a schematic view of a backside of a third pod, in accordance with embodiments of the present invention;

FIG. 8 depicts three removable accessories that are each capable of being inserted into pod, in accordance with embodiments of the present invention;

FIG. 9 depicts a schematic view of a pod without an accessory inserted therein, in accordance with embodiments of the present invention;

FIG. 10A depicts a first side of an accessory, in accordance with embodiments of the present invention;

FIG. 10B depicts a second side of the accessory, in accordance with embodiments of the present invention;

FIG. 11 depicts a schematic view of a sensor of the accessory integrated with the body portion, in accordance with embodiments of the present invention;

FIG. 12 depicts a schematic view of the sensor of the accessory integrated with the body portion, with a reverse flow direction, in accordance with embodiments of the present invention;

FIG. 13 depicts a schematic view of the pod with an accessory inserted therein, in accordance with embodiments of the present invention;

FIG. 14 depicts a schematic view of the pod with an accessory inserted therein and a cover covering the accessory, in accordance with embodiments of the present invention;

FIG. 15 depicts a flowchart of a method for automatic shutoff of gas supply to a patient worn device, in accordance with embodiments of the present invention;

FIG. 16 depicts a pod system incorporated into various objected in an environment, in accordance with embodiments of the present invention;

FIG. 17 depicts a front view of a pod system according to an exemplary embodiment of the present invention;

FIG. 18 depicts a perspective view of the pod system shown in FIG. 17, with a cover 60 installed onto the pods, in accordance with embodiments of the present invention;

FIG. 19 depicts a perspective view of another embodiment of a pod system, in accordance with embodiments of the present invention;

FIG. 20 depicts a front view of the pod system shown in FIG. 19, in accordance with embodiments of the present invention;

FIG. 21 depicts a bottom, perspective view of an accessory and a pod before insertion of the accessory into the pod, in accordance with an exemplary embodiment of the present invention;

FIG. 22 depicts a perspective view of an accessory and a pod before insertion of the accessory into the pod, in accordance with an exemplary embodiment of the present invention;

FIG. 23 depicts a connection between an accessory and a pod, in accordance with an exemplary embodiment of the present invention;

FIG. 24 depicts a first type of accessory, in accordance with an exemplary embodiment of the present invention;

FIG. 25 depicts a second type of accessory, in accordance with an exemplary embodiment of the present invention; and

FIG. 26 depicts a third type of accessory, in accordance with an exemplary embodiment of the present invention.

### DETAILED DESCRIPTION

A detailed description of the hereinafter described embodiments of the disclosed apparatus and method are presented herein by way of exemplification and not limitation with reference to the Figures. Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure.

As a preface to the detailed description, it should be noted that, as used in this specification and the appended claims, the singular forms “a”, “an” and “the” include plural referents, unless the context clearly dictates otherwise.

In brief overview, SOT solutions include medical gas flow regulators and suction regulators that attach to existing medical gas outlets on a wall of a hospital room. Conventional SOT devices protrude from the wall because the flow regulator mechanism is located external from the wall, which leads to several problems. Incidental contact with the SOT devices extending outward from the wall can damage the devices. Clinicians remove the SOT devices from the wall in a hospital room to use in another room and thus the current hospital room is left without a critical SOT device. Further, tubes are attached to SOT devices that connect the SOT device to a patient worn device for delivering medical gas, oxygen, vacuum, etc. to a patient. If the tube is disconnected from the SOT device, a supply of gas can enter the room without anyone knowing. The flow of gas into a room leads to a waste of medical resources and potentially poses other problems if the flow of gas continues without corrective action taken. If the tube is disconnected during a suction operation, the vacuum pump continues to operate without drawings medium/gas back through the system.

Embodiments of the present invention utilize a sensor for detecting a pressure that indicates a loose or interrupted connection between the SOT device and the patient worn device. If the sensor detects a pressure drop (e.g. a change in pressure) to atmospheric pressure, the flow of the gas is automatically shut off or otherwise discontinued to prevent further flow of the gas into the environment.

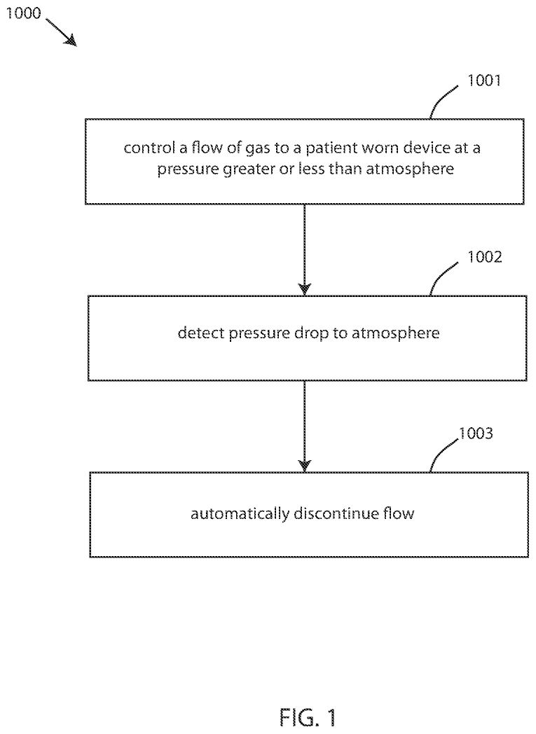

Referring now to the drawings, FIG. 1 depicts a flowchart of a method 1000 for automatically discontinuing a flow of gas, in accordance with embodiments of the present invention. The method 1000 can be implemented with conventional SOT devices or with a pod system described below. Step 1001 controls a flow of gas to a patient worn device at a pressure greater or less than atmospheric pressure. The flow of gas to the patient worn device (e.g. mask or rebreather) is typically established by a tube that connects to an outlet of the SOT device and the patient worn device. One or more valves of a flow regulator are opened to allow the flow of gas to the patient worn device, from the outlet of the SOT through the tube to the patient worn device. The flow regulator of the SOT device controls the flow of gas to the patient worn device, for example, at a specific flow rate or pressure greater than atmospheric pressure. For suction operations, the flow regulator of the SOT device controls the vacuum at a pressure less than atmospheric pressure.

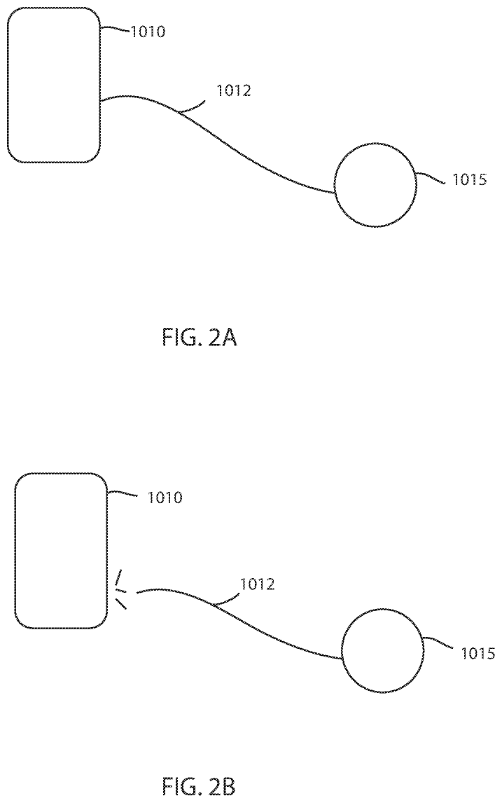

FIG. 2A depicts a schematic view of an SOT device 1010 connected to a patient worn device 1015 via tubing 1012, in accordance with embodiments of the present invention. SOT device 1010 can be a conventional SOT device that protrudes from the wall (i.e. flow regulator mechanism is located external from the wall) or can be a pod or pod system described herein that is recessed behind the wall. A conventional SOT device may need to be fitted with a controller, one or more sensors, actuator, for example, to turn the valve, in response to a pressure drop to atmospheric pressure.

During normal operation, a fluidic connection between the SOT device 1010 and the patient worn device 1015 is not interrupted or otherwise hindered, and the pressure at the outlet of the SOT device 1010 is consistent, at least within a close range, of the set pressure of the flow regulator of the SOT device 1010. For example, if the set pressure delivered to the patient is in a range of 2-10 cm H20 (0.03-0.14 psi), the set pressure will be maintained as long as the tubing 1012 is connected. The outlet of the SOT device 1010 is not open to the atmosphere.

FIG. 2B depicts a schematic view of the SOT device 1010 disconnected from the patient worn device 1015, in accordance with embodiments of the present invention. During use of the SOT device 1010, the tubing 1012 may be disconnected, partially or fully, from the SOT device 1010. For example, a movement of a patient may result in the tube 1012 pulling off of the outlet/connection point at the SOT device 1010. If the connection between the SOT device 1010 and the patient worn device 1015 becomes disconnected, a pressure at the SOT device 1010 goes to atmospheric pressure because the outlet of the SOT device 1010 is now open to the atmosphere. For example, if the set pressure delivered to the patient is in a range of 2-10 cm H20 (0.03-0.14 psi), and the tubing 1012 is disconnected, the set pressure will be change to atmospheric pressure.

Returning to the method 1000 of FIG. 1. step 1002 detects the pressure drop to atmospheric pressure. For instance, when the tube 1012 is disconnected from the SOT device 1010, the pressure drop to atmosphere is detected by a pressure sensor that can detect a pressure differential between a pressure upstream of the outlet of the SOT device 1010 and downstream of the outlet of the SOT device 1010. In response to the detection of the pressure drop to atmosphere, step 1003 automatically discontinues the flow of the gas to the patient worn device 1015. The flow is stopped by closing of one or more valves of the SOT device 1010. The valve(s) may be closed automatically via actuation of the valve electronically by a flow management device of the SOT device 1010. In an exemplary embodiment, a control interface, a controller, a hardware processor, and the like, which is in communication with the pressure sensor of the SOT device 1010, sends a control signal to the valve(s) to close, thereby preventing gas to flow to the patient worn device 1015 or stopping a vacuum operation.

While the method 1000 can be implemented with conventional SOT devices or with a pod system described below, embodiments of a method for automatically discontinuing a flow of gas will now be described with respect to implementation of the method with a pod system. The pod system includes one or more pods capable of receiving a removable pod accessory. A pressure sensor is part of the pod accessory removably inserted into a pod that is recessed inside a wall (or a headwall of a bed unit). Each pod of the pod system has a flow management device (e.g. oxygen, medical air, vacuum, etc.) that fits within a rough-in box behind the wall. The connections are made within the recess of the wall. The pods have a built-in flow management device (e.g. electronic solenoid valve) that regulates the flow of gas in both directions through the pod from a source. Each pod is recessed into the wall and includes a control unit with a touch screen that allows a clinician to adjust the flow/suction using the touch screen. The pods also include a portion that allows a mechanical connection to a pod frame, with a port that allows for an attachment of tubing to go from the pod to the patient. Various accessories can be removably attached to the pod, establishing fluidic communication with a fluid source located remotely. For example, a casing of the pod includes structure for easy attachment of the accessories to the pod, which can be slid into the pod shell and locked into place. The accessories include a pressure sensor for detecting a pressure drop, a pressure differential, or any pressure value that indicates a connection from the flow management device to the patient worn device is has been disconnected. The sensor of the accessory communicates with the flow management device, which takes corrective action to automatically discontinue the flow of gas, such as actuating/closing a valve associated with the flow management device. Thus, if a connection between an SOT device and a patient worn device is disconnected, the flow of gas into a room environment is stopped immediately and without requiring action by the clinician to notice that the tube is disconnected and/or to manually close a valve.

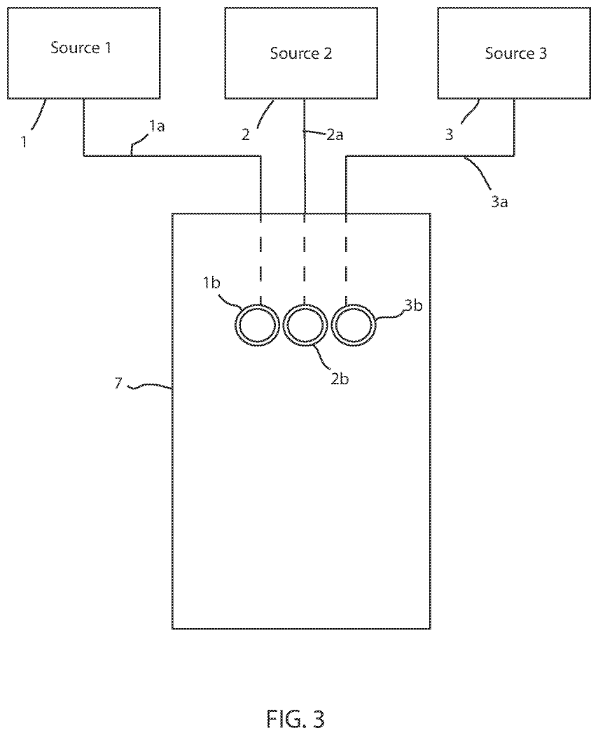

FIG. 3 depicts a schematic view of a rough-in assembly 7 having connections to a plurality of sources 1, 2, 3, in accordance with embodiments of the present invention. Rough-in assembly 7 is located behind a finished surface, such as a wall of a hospital room, and is configured to be attached to one or more structures located behind the finished surface, such as metal or wood wall construction elements. Rough-in assembly 7 is a receptacle safe for in-wall or covered applications that has an open face to accommodate one or more pods of a pod system, as described in greater detail infra. The rough-in assembly 7 is sized and dimensioned to allow a pod to fit therein. In an exemplary embodiment, a depth of the rough-in assembly 7 can be large enough so that a pod fits entirely within the rough-in assembly 7 so that a front surface of the pod can be flush or recessed with a finished surface. In another embodiment, the depth of the rough-in assembly 7 can be reduced such that only a portion of the pod fits within the rough-in assembly 7 so that a front surface of the pod protrudes slightly from the finished surface.

The rough-in assembly 7 accommodates connections to sources 1, 2, 3 located remote from the rough-in assembly 7. For instance, rough-in assembly 7 safely and securely accommodates fluidic couplings 1b, 2b, 3b of the sources 1, 2, 3 within the rough-in assembly 7. In the illustrated embodiment, supply lines 1a, 2a, 3a associated with a first source 1, a second source 2, and a third source 3, respectively enter the rough-in assembly 7 through a back wall of the rough-in assembly 7; however, the supply lines 1a, 2a, 3a may be directed through any surface of the rough-in assembly 7. The supply lines 1a, 2a, 3a pass through openings either pre-formed or created in the field, such as by removing knockouts on the rough-in assembly 7. The fluidic couplings 1b, 2b, 3b are devices configured to couple or connect two fluidic channels together. A type and/or size of the fluidic coupling 1b, 2b, 3b depends on the source 1, 2, 3. Examples of fluidic couplings 1b, 2b, 3b include a fitting, a connector, an adapter, check valves, hose barbs, elbows, quick-connect, etc. After installation of the rough-in assembly 7 and fluidic couplings 1b, 2b, 3b within the rough-in assembly 7, access to any of the sources 1, 2, 3 is possible, depending on a type of pod inserted within the rough-in assembly 7, as described in greater detail infra.

While three sources are depicted in FIG. 3, less than three or more than three sources can be roughed in to rough-in assembly 7. The sources 1, 2, 3 can be tanks, reservoirs, vessels, containers, and the like, storing a gas, fluid, or mixture of gases, with a mechanism for delivering the gas, fluid, or mixture from their remote location via supply lines 1a, 2a, 3a. A non-exhaustive list of the types of sources 1, 2, 3 includes medical air (Med Air), carbon dioxide (CO2), helium (He), nitrogen (N2), nitrous oxide (N20), oxygen (O2), oxygen/carbon dioxide mixture (O2/C02 n %, n is % of CO2), medical-surgical vacuum (Med Vac), waste anesthetic gas disposal (WAGD), non-medical air (Level 3 gas-powered device), laboratory air, laboratory vacuum, instrument air, and other mixtures having a ratio of Gas A/Gas B in percentage. Further, reference to SOT is a term intended to be inclusive of gases that contain oxygen and that do not necessarily include oxygen.

In FIG. 3, the first source 1 is a source of oxygen, the second source 2 is a source of medical air, and the third source 3 is a vacuum source. The first source 1 is accessible via the fluidic coupling 1b within the rough-in assembly 7 via supply line 1a, which is connected to the first source 1 at one end and connected to the fluidic coupling 1b at the other end. Similarly, the second source 2 is accessible via the fluidic coupling 2b within the rough-in assembly 7 via supply line 2a, which is connected to the second source 2 at one end and connected to the fluidic coupling 2b at the other end, and the third source 3 is accessible via the fluidic coupling 3b within the rough-in assembly 7 via supply line 3a, which is connected to the third source 3 at one end and connected to the fluidic coupling 3b at the other end. Supply lines 1a, 2a, 3a are located behind a finished surface and can have various lengths and intermediate fittings to accommodate runs from the source to the rough-in assembly 7. The supply lines 1a, 2a, 3a are terminated by the fluidic couplings 1b, 2b, 3b inside the rough-in assembly 7. Located at least partially within a casing 10 of the pod 100. Alternatively, the supply lines 1a, 2a, 3a are terminated outside of the rough-in assembly 7, behind the finished surface, with an intermediate connection line completing the connection from the ends of the supply line 1a, 2a, 3a to the fluidic couplings 1b, 2b, 3b within the rough-in assembly 7; the intermediate connection line can be a flexible tubing that provides flexibility to connect the sources to rough-in assembly 7 in certain construction environments where existing piping from the sources is connected to a new installation of the rough-in assembly 7.

Because the rough-in assembly 7 houses fluidic connections to multiple sources 1, 2, 3, any of the sources 1, 2, 3 can be accessed at the location of the rough-in assembly 7. The fluidic couplings 1b, 2b, 3b within the rough-in assembly 7 are discrete and separate from each other so that a connection can be made to one fluidic coupling only and not the others; thus, only gas from the source associated with the fluidic coupling which has a mated connection flows through the pod system, while the other fluidic couplings without a mated connection prevent a flow of the gas from the sources into a room environment. Which source 1, 2, 3 is accessed depends on which pod is inserted into the rough-in assembly 7.

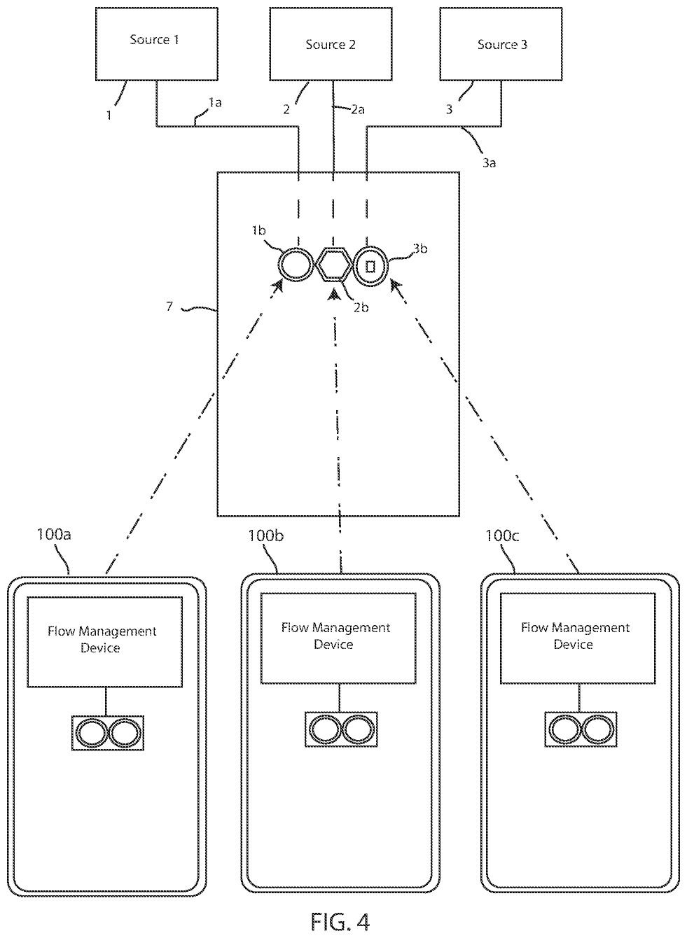

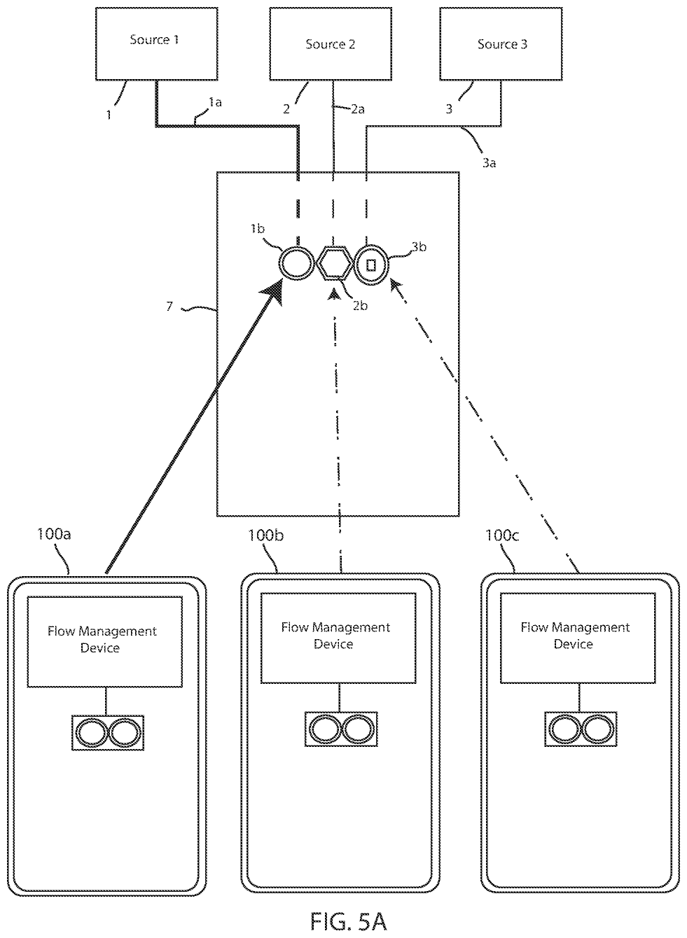

FIG. 4 depicts a schematic view of pods insertable into the rough-in assembly 7 of FIG. 3, in accordance with embodiments of the present invention. Each pod is specific to a type of gas associated with a source 1, 2, 3. For example, pod 100a is specifically designed for managing a flow of gas associated with source 1, pod 100b is specifically designed for managing a flow of gas associated with source 2, and pod 100c is specifically designed for managing a flow of gas associated with source 3. If access to source 1 (e.g. oxygen) is desired, then pod 100a can be inserted into rough-assembly 7, as shown in FIG. 5A. If access to source 2 (e.g. medical air) is desired, then pod 100b can be inserted into rough-assembly 7, as shown in FIG. 5B. If access to source 3 (e.g. vacuum) is desired, then pod 100c can be inserted into rough-assembly 7, as shown in FIG. 5C. Each pod 100a. 100b, 100c is removably inserted into the rough-in assembly 7 so that the pod 100a can be removed and swapped with a different pod 100b or 100c, at a same location within the room environment. Because the pods can be swapped out, a clinician can access different types of gases at the same location in the wall, for example, which makes the hospital room more flexible to meet various needs of a medical facility.

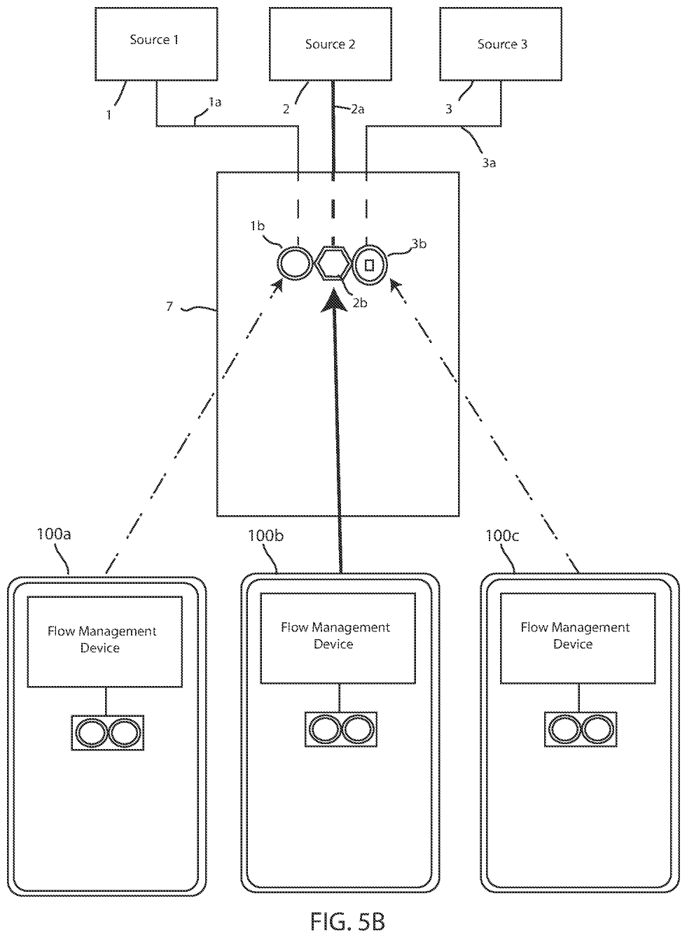

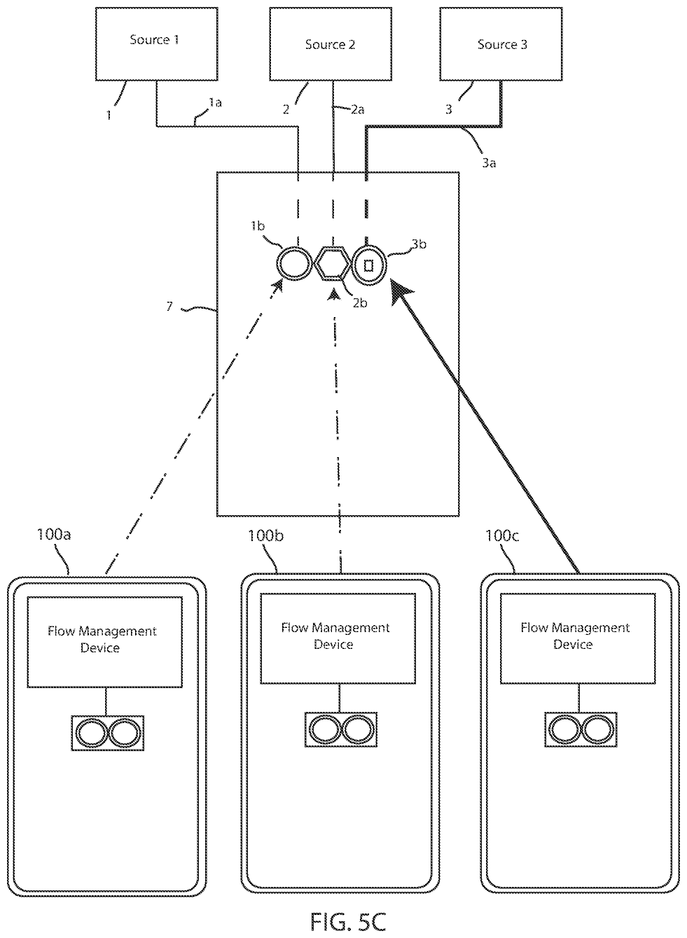

FIG. 6 shows a schematic view of a pod 100 inserted within the rough-in assembly 7, in accordance with embodiments of the present invention. As shown, pod 100 fits within an interior of the rough-in assembly 7; pod 100 could be pod 100a, 100b, 100c, or any pod having a specific type of flow management for use with a specific source. As a function of inserting the pod 100 into the rough-in assembly 7, a fluidic coupling(s) on the back of the pod 100 mates with one of the fluidic couplings 1b, 2b, or 3b that corresponds to the fluidic coupling on the pod 100. FIGS. 7A-7C depict a schematic view of a backside of the pods 100a, 100b, 100c, respectively, in accordance with embodiments of the present invention. The pods 100a, 100b, 100c include a fluidic coupling 11a, 11b, 11c on a back surface 12 of the pod that is designed to mate with one of the fluidic couplings 1a, 1b, 1c. The fluidic couplings 11a, 11b, 11c are designed to mate with one of the fluidic couplings 1a, 1b, 1c based on one or more different structural configurations and/or industry standardized coupling connections. For instance, the shape of the couplings, the location of the couplings, a pin index of the couplings, a diameter of the couplings, etc. can be used to control which couplings 11a, 11b, 11c of the pod can be coupled to the couplings 1a, 1b, 1c of the rough-in assembly 7. In an exemplary embodiment shown in FIGS. 7A-7C, the fluidic couplings 11a, 11b, 11c each have a unique shape (shown schematically) that correspond to a unique shape of the fluidic couplings 1b, 2b, 3b (shown schematically) to indicate that one type of fluidic coupling of the pod is configured to mate with one type of fluidic coupling within the rough-in assembly 7, associated with a specific source of gas.

The mated connected between fluidic coupling 1b, 2b, 2c and 11a, 11b, 11c of the pod allows a flow of gas to a flow management device 20 of the pod 100, which is located within a casing 10 of the pod 100. The flow management device 20 is configured to manage, regulate, or otherwise control a flow of gas through the pod 100, coming from one of the plurality of sources 1, 2, 3. Examples of the flow management device 20 are a flow meter or flow regulator that includes at least one electronic valve for managing a flow of gas through the pod 100. The specific design and construction of the flow management device 20 depends on which type of gas the pod is designed to regulate. For instance, a pod may include a type of flow management device for managing the flow of oxygen, and would also include a fluidic coupling on the back of the pod for mating with the fluidic coupling 1b associated with the first source 1. A pod may include a type of flow management device for managing the flow of medical air, and would also include a fluidic coupling on the back of the pod for mating with the fluidic coupling 2b associated with the second source 2. A pod may include a type of flow management device for managing a vacuum for suction through pod, and would also include a fluidic coupling on the back of the pod for mating with the fluidic coupling 3b associated with the third source 3. Examples of flow management devices include an electronic needle valve and controller, and an integrated mass flow control valve.

A control interface 25 is electrically coupled to the flow management device 20 for controlling at least one function of the flow management device 20. For instance, the control interface 25 includes a controller and a display, and may utilize various input methods such as a touch screen, button interface, and/or rotary dial that allows clinicians to input commands to the flow management device 20 via touch or button press. The control interface 25 sends instructions/commands to the flow management device 20 to perform at least one function, such as maintain a certain flow rate, increase a flow rate, decrease a flow rate, open a valve, close a valve, etc. In this way, a clinician can conveniently control the flow management device 20 via the control interface 25 to treat a patient with SOT as required. The control interface 25 is in communication with a pressure sensor of accessory via receptacle 38. The connection between the receptacle 38 and the control interface is shown in a lighter solid line in FIG. 6. When the accessory with the pressure sensor is inserted into the pod 100, the sensor mates with the receptacle 38 to establish an electrical connection (i.e. wired, direct connection) between the sensor and the control interface 25. The receptacle 38 is built into the wall of the pod 100 and may be located at various points within the pod 100, depending on the design of the pod and the removable accessory insertable therein. In addition to establishing an electrical connection for transmission of data (i.e. communication), the receptacle 38 may also be configured to supply power to the pressure sensor of the accessory when the accessory is fully inserted into the pod 100. The receptacle 38 may be a Universal Serial Bus interface, such as USB-C, or equivalent interface. The receptacle 38 can also be other types of connection ports that are capable of establishing communication with the control interface 25 and supplying power to the pressure sensor.

In an exemplary embodiment, the receptacle 38 is located proximate or otherwise near the at least one fluidic coupling 30 within the pod 100 so that an electrical connection on the accessory can be disposed proximate the fluidic connections on the accessory for mating with the fluidic connection 30 the flow management device 20.

Managed or regulated gas flows between the flow management device 20 and at least one fluidic coupling 30 located at least partially within the casing 10 of the pod 100. The fluidic coupling 30 is fluidically connected to the flow management device 20, and, when the pod 100 is inserted within the rough-in assembly 7, the fluidic coupling 30 is also fluidically connected to the supply line and source specific to the pod 100. By operation of the flow management device 20, gas flows from the source through the supply line and through the flow management device 20 to the coupling 30 in a controller manner. The at least one fluidic coupling 30 is a fitting, a connector, an adapter, check valves, hose barbs, elbows, quick-connect, etc. that is configured to mate with a fluidic coupling an accessory insertable within the pod 100. The accessory insertable within the pod includes an outlet though which the regulated gas can flow in or out of the accessory and to the patient via tubing connected to the outlet.

The control interface 25 of pod 100 may generally comprise a processor, an input device coupled to the processor, an output device coupled to the processor, and memory devices each coupled to the processor. The input device, output device and memory devices may each be coupled to the processor via a bus. Processor may perform computations and control the functions of the pod system, including executing instructions included in computer code for the tools and programs capable of implementing a method for automatically discontinuing a gas supply to a patient worn device, wherein the instructions of the computer code may be executed by processor via memory device. The computer code may include software program instructions that may implement one or more algorithms for implementing methods and functions of the pod system, as described in detail above. The processor executes the computer code. Processor may include a single processing unit locally resided within the pod housing, or may also be distributed across one or more processing units in one or more locations (e.g., on a client and server).

The memory device of the control interface 25 may include input data that includes any inputs required by the computer code. The output device displays output from the computer code. The memory devices may be used as a computer usable storage medium (or program storage device) having a computer-readable program embodied therein and/or having other data stored therein, wherein the computer-readable program comprises the computer code. Generally, a computer program product (or, alternatively, an article of manufacture) of the computer system may comprise said computer usable storage medium (or said program storage device).

Memory devices of the control interface 25 include any known computer-readable storage medium. In one embodiment, cache memory elements of memory devices may provide temporary storage of at least some program code in order to reduce the number of times code must be retrieved from bulk storage while instructions of the computer code are executed. Moreover, similar to processor, memory devices may reside at a single physical location, including one or more types of data storage, or be distributed across a plurality of physical systems in various forms. Further, memory devices can include data distributed across, for example, a local area network (LAN) or a wide area network (WAN). Further, memory devices may include an operating system.

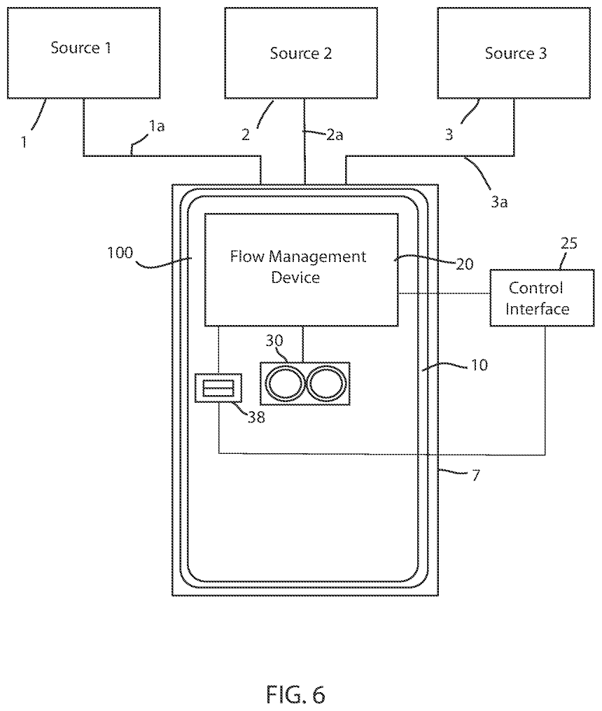

As will now be described with reference to FIG. 8, an accessory can be insertable within the pod 100. The accessory is configured to fluidically and mechanically connect to the fluidic coupling 30 built into the pod 100 so that the gas flows through the accessory and out to the patient. One accessory is insertable into the pod 100 at a given time, but there are many different accessories that can be used with pod 100. The accessory resides within the pod casing 10 when inserted or otherwise attached to the pod 100, and is removable from the pod 100 so that a new accessory can be inserted into the pod 100. FIG. 8 shows three different, removable accessories 5, 6, 7 that are each capable of being inserted into pod 100 but many different accessories can be designed to fit within pod 100. Each accessory 5, 6, 7 includes at least one fluidic coupling 5a, 6a, 7a configured to mate with fluidic coupling 30 of the pod 100 and a pressure sensor configured to mate with receptacle 38 of the pod 100, but each accessory has a different function and/or feature that provides flexibility to the clinician.

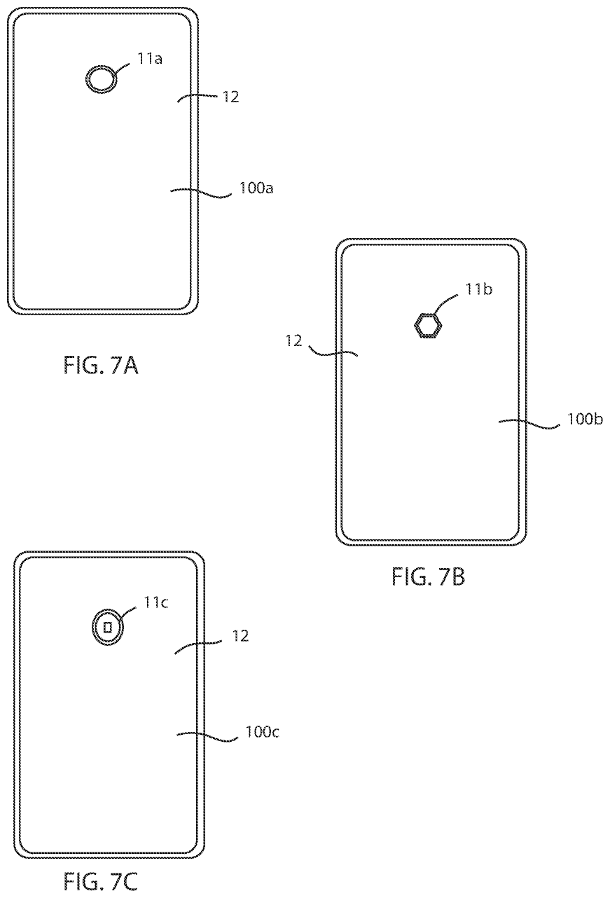

FIG. 9 depicts a schematic view of a pod 100 without an accessory inserted therein, in accordance with embodiments of the present invention. The pod 100, as illustrated in FIG. 9, includes a pod casing 10 that defines an interior region 14 of the pod 100. The casing 10 includes two side walls and back surface (not shown in FIG. 9), and open face to allow insertion of an accessory and installation of the flow management device. The flow management device is located behind the control interface 25 so that a clinician interfaces with the a UI of the control interface 25 and not the flow management device disposed within the pod 100. The control interface 25 is shown with a plurality of graphical icons and/or buttons for clinician interaction with the pod 100, such as increasing or decreasing a flow rate through the pod 100. The fluidic coupling 30 is disposed within the interior region 14 and is accessible through the open front face of the casing 10 for inserting and removing an accessory. Likewise, receptacle 38 is disposed within the interior region 14 and is accessible through the open front face of the casing 10 for inserting and removing an accessory.

Moreover, the pod 100 includes an accessory receiving mechanism 50 disposed within the casing 10 of the pod 100. The accessory receiving mechanism 50 facilitates an insertion and fluidic coupling of the accessory within the pod casing 10; the accessory receiving mechanism 50 also facilitates the electrical connection between the pressure sensor of the accessory and the pod 100. The accessory receiving mechanism 50 includes a first receiving structure 51 on a first side of the pod casing 10 and a second receiving structure 52 on a second side opposite the first side of the pod casing 10. The first receiving structure 51 and the second receiving structure 52 extend towards the rear wall of the casing 10, starting proximate or at the front of the casing 10. In an exemplary embodiment, the first receiving structure 51 and the second receiving structure 52 are each a lip that protrudes from a housing/enclosure of the sensor 40 that guides the accessory into the casing 10 and into proper mating position with the fluidic coupling 30 and receptacle 38 of the pod, while supporting, at least partially, the weight of the accessory when inserted and mated within the pod 100. The accessory receiving mechanism 50 is positioned within the pod 100 at a height within the casing 10 to align a fluidic coupling, such as fluidic couplings 5a, 6a, 7a of accessories 5, 6, 7 within the fluidic coupling 30 disposed within the pod casing 10. Thus, the location of the accessory receiving mechanism 50 within the pod 100 can vary across different pod designs and dimensions.

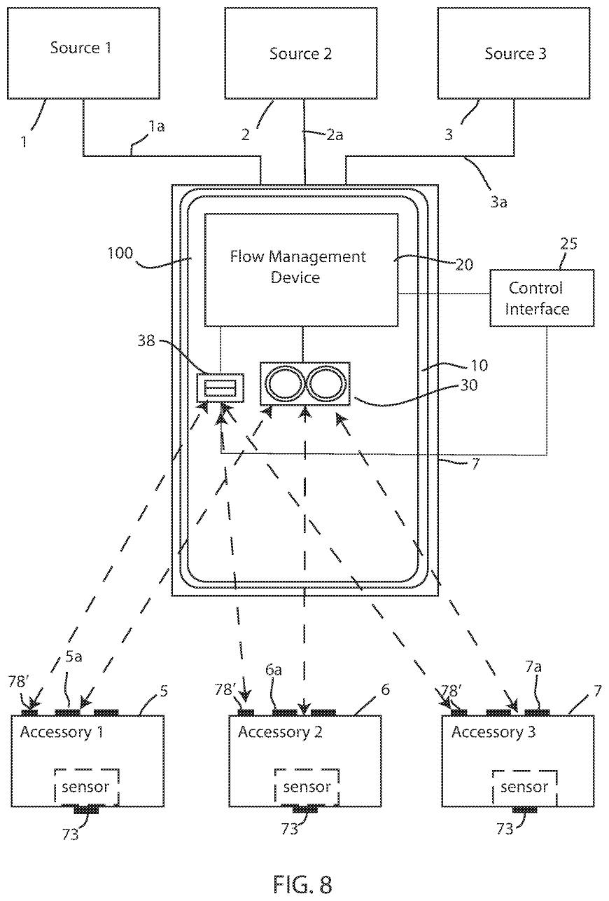

FIGS. 10A-10B depict a schematic view of accessory 70, in accordance with embodiments of the present invention. Accessory 70 could be accessory 5, accessory 6, accessory 7, or any accessory having a specific function or structure, and includes a pod engagement mechanism 76 that universally cooperates with accessory receiving mechanisms 50 of pod 100. The removable accessory 70 includes a body portion 75 having a first side 70a and a second side 70b. The body portion 75 may be a lid or cover that is a solid piece of material capable of being machined or manufactured to include coupling 71 and an outlet 73. The accessory 70 includes a pod engagement mechanism 76 disposed on the body portion 75. The pod engagement mechanism 76 includes a first engagement structure at a first location on the body portion 75 and a second engagement structure at a second location of the body portion, opposite the first location. The first engagement structure and the second engagement structure of the pod engagement mechanism 76 is a protrusion or other lip or extension of the body portion 75 that is configured to cooperate with the accessory receiving mechanism 50 of the pod 100.

Moreover, the accessory 70 incudes a canister 74 operably attached to the body portion 75. The canister 74 may be removably attached to the body portion 75 so that the canister 74 of the accessory 70 is further customizable. For instance, the canister 74 may be threadably attached to the body portion 75 so that the canister 74 is easily removed and replaced with a canister 74 of different size or function. The canister 74 may be removably attached to the lower side of the body portion 75 and can be twisted on or off to replace the canister with a new canister of same or different shape/type. The canister 74 is a container or storage device that has an interior space that allows a flow of gas between the fluidic coupling 71 that mates with the fluidic coupling 30 of the pod 100 and the outlet 73. The interior space of the body portion 74 may store a fluid, such as water, for humidifying the gas flowing through the accessory. The interior space of the body portion 74 may also store waste fluid/material drawn into the outlet 73 of the accessory 70.

FIG. 10A depicts the first side 70a of the accessory 70, and FIG. 10B depicts the second side 70b of the accessory 70. The first side 70a of the accessory 70 faces toward a rear wall of the pod 100 when the accessory 70 is removably inserted within the pod casing 10. Fluidic couplings 71 are disposed on the body portion 75 at the first side 70a of the accessory 70 and are configured to mate with the fluidic coupling 30 of the pod 100 when the accessory 70 is removably inserted into the pod 100. While two fluidic couplings 71 are shown in FIG. 10A, there may be a single fluidic coupling that mates with a single fluidic coupling 30 inside the pod 100. Further, while the couplings are depicted as extending from the side of the accessory, the couplings could be made from the top of the accessory as well. The mating of the fluidic couplings 71, 30 establish fluidic communication between the flow management device 20 of the pod 100 and the accessory 70. For instance, at least one fluidic coupling 71 is configured to be fluidically coupled to a flow management device 20 of the pod 100, as a function of the removable accessory 70 being inserted within the pod casing 10 of the pod 100.

The second side 70b of the accessory 70 faces away from a rear wall of the pod 100 when the accessory 70 is removably inserted within the pod casing 10. An outlet 73 is disposed on the body portion 75 at the second side 70b of the accessory 70 and is configured to be accessible by a clinician when the accessory 70 is removably inserted into the pod 100, for attaching tubing that facilitates gas flow between the pod and the patient worn device. While one outlet 73 is shown in FIG. 10B, there may be a more than outlet disposed on the accessory 70. The outlet 73 is in fluidic communication with the flow management device 20 of the pod 100. The fluid communication may be a dedicated flow path from the fluidic coupling 71 to the outlet 73, or may pass generally through the interior space of the canister 74.

Moreover, the accessory 70 includes a pressure sensor 78. The pressure sensor 78 may be a pressure differential sensor, differential pressure transducer, a bidirectional difference pressure sensor, etc. The sensor 78 is disposed proximate the outlet 73 and configured to detect a pressure differential at the outlet 73. In an exemplary embodiment, the sensor 78 is integrated into the body portion 75, such as lid of the accessory 70. The pressure sensor 78 is electrically coupled to an electrical connector 78′ that at least partially protrudes from the body portion 75 for insertion into the receptacle 38 of the pod 100. In an exemplary embodiment, the electrical connector 78′ may be a male-type connector that is configured to mate with a female-type connector, such as receptacle 38. The electrical connector 78′ may be a USB type connector or similar electrical connection interface that establishes an electrical connection with the receptacle 38 of the pod 38 for transmitting data and receiving power to power the pressure sensor 78. The act of inserting the accessory 70 into the pod establishes the fluidic connections and the electrical connection with the pod, and pressure readings captured by the pressure sensor 78 can be transmitted to the pod 100 via the electrical and mechanical connection between the electrical connector 78′ and the receptacle 38. In an alternative embodiment, the sensor 78 may be wirelessly connected to the control interface 25, for example, via a short range communication network or a wireless local area network, or can be wired directly to the control interface 25.

The control interface 25 sends a control signal to a flow control device to automatically stop the flow of gas, in response to detecting a pressure drop to atmospheric pressure. The flow control device is a device that can be actuated by the control interface 25 to allow or prevent a flow of gas or a suction operation. Examples of a flow control device include a valve, a flow control valve, a needle valve, a globe valve, gate valve, pinch valve, diaphragm, valve, check valve, solenoid valve, an orifice, and the like.

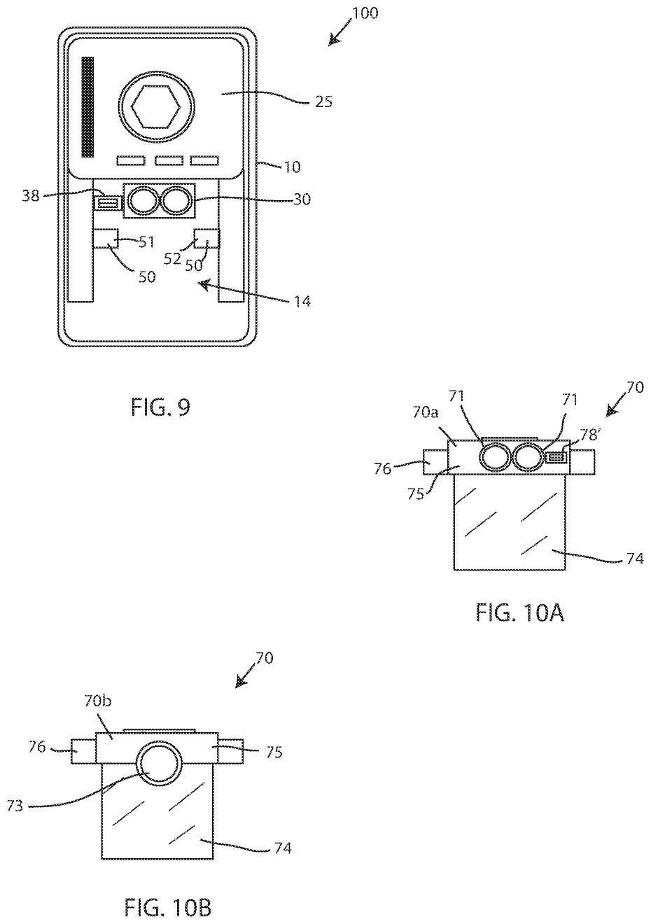

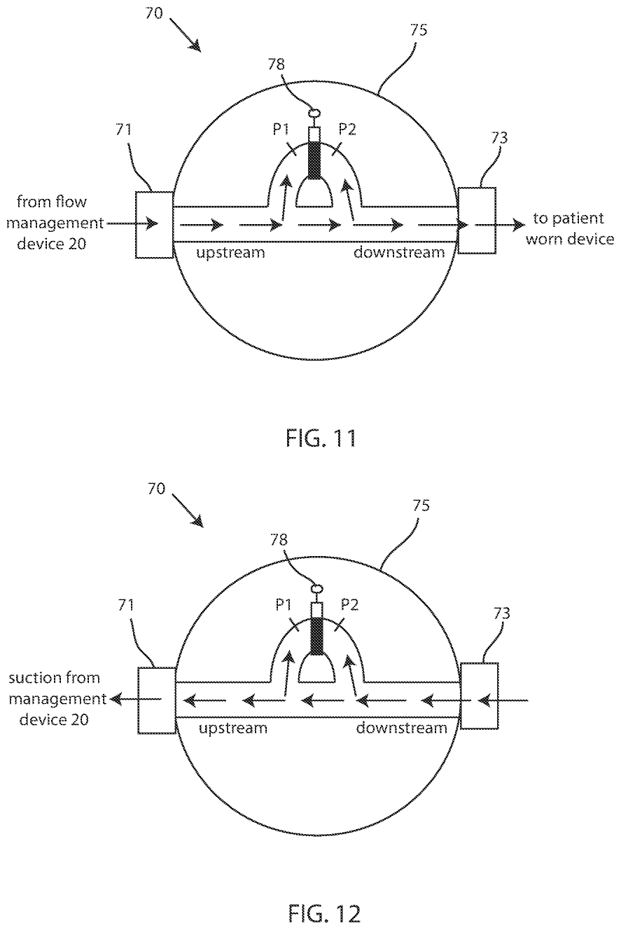

FIG. 11 depicts a schematic view of the sensor 78 of the accessory 70 integrated with the body portion 75, in accordance with embodiments of the present invention. In the illustrated embodiment, a flow of gas is depicted by arrows from the flow management device 20 to the patient worn device. The fluidic coupling 71 is connected to the outlet 73 by a fluidic pathway. The fluidic pathway includes a main pathway 79a that extends directly from the fluidic coupling 71 to the outlet 73, a first secondary pathway 79b upstream of the sensor 78 and a second secondary pathway 79c downstream of the sensor 78. The first secondary pathway 79b allows for a portion of the gas upstream of the sensor 78 (i.e. upstream pressure) to flow to P1, or the high side, of the pressure sensor 78. The second secondary pathway 79c allows for a portion of the gas downstream of the sensor 78 (i.e. downstream pressure) to flow to P2, or the low side, of the pressure sensor 78. If the downstream pressure decreases to or close to atmospheric pressure as a result of the fluidic connection between the flow management device 20 and the patient worn device being disconnected or largely interrupted, for example, by the tubing being physically removed from the outlet 73, the sensor 78 will detect the pressure differential between P1 and P2. The sensor 78 outputs a signal to or the signal output is read by the control interface 25 of the flow management device 20 that a pressure differential indicating a drop in downstream pressure has been detected. FIG. 12 depicts a schematic view of the sensor 78 of the accessory 70 integrated with the body portion 75 for a suction operation, in accordance with embodiments of the present invention. The sensor 78 largely operates in the same manner as described above but the flow is in a reverse direction.

When the control interface 25 of the pod 100 discontinues the flow of gas to the patient worn device, or stops the suction operation, the control interface 25 can notify a clinician with at least one of an audible notification, a visual notification on a graphical user interface, an indicator, a transmission to a remote computer, or a combination thereof.

The audible notification may be an alarm that makes a noise through a speaker electrically coupled to the control interface 25; the speaker can be a built-in speaker of the pod 100. The purpose of the alarm is to alert a clinician to the need to reattach the tubing of the patient worn device to the pod 100. In practice, the clinician will hear the alarm and follow training and appropriate rules and regulations for either reattaching the tubing or replacing the tubing, etc.

The visual notification may be a light, LED, or graphic that activates on a graphical user interface associated with the pod 100 either built-in with the pod 100 or located proximate the pod 100, that emits a light conspicuous enough to be noticed by a clinician. The purpose of the visual notification is to alert a clinician to the need to reattach the tubing of the patient worn device to the pod 100. In practice, the clinician will see the visual notification and follow training and appropriate rules and regulations for either reattaching the tubing or replacing the tubing, etc.

The indicator may be a light bulb or LED located within the hospital room, on the patient bed, computer stations external to the patient's room, wirelessly connected or wired to the control interface 25 of the pod 100, configured to emit a light conspicuous enough to be noticed by a clinician. The purpose of indicator is to alert a clinician to the need to reattach the tubing of the patient worn device to the pod 100. In practice, the clinician will see the visual notification and follow training and appropriate rules and regulations for either reattaching the tubing or replacing the tubing, etc.

The transmission to a remote computer may be a signal transmitted over the Internet to a computer, tablet, laptop, etc. being monitored by a clinician. The purpose of the transmission to a remote computer is to alert a clinician to the need to act, even if the clinician is not physically present in the patient room, near the patient's room, or not at a dedicated clinician station. In practice, the clinician will see the transmission and follow training and appropriate rules and regulations for either reattaching the tubing or replacing the tubing, etc.

The control interface 25 of the pod 100 can be programmed, for example, via user interface, to trigger one type of notification or a combination of types of notifications to work in concert in alerting a clinician. The control interface 25 can trigger the audible notification, the visual notification on a graphical user interface, the indicator, and/or the transmission to the remote computer, in response to the signal output by the sensor 78.

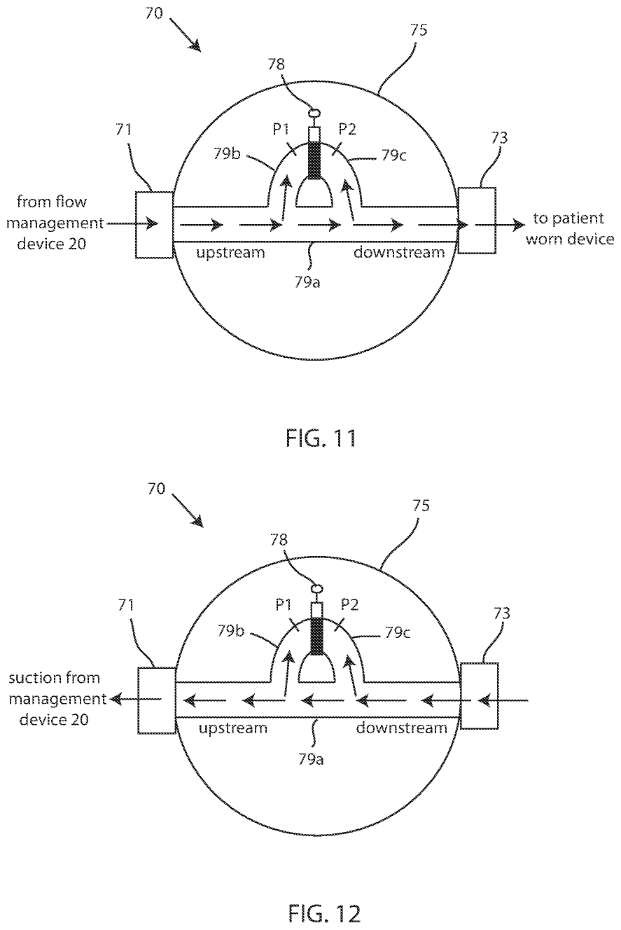

FIG. 13 depicts a schematic view of the pod 100 with an accessory 70 inserted therein, in accordance with embodiments of the present invention. In the illustrated embodiment, the pod 100 is shown with accessory 70 disposed within the pod 100. As can be seen in FIG. 13, the accessory 70 resides within the interior region 14 of the casing 10 when inserted into the pod 100, and the pod engagement mechanism 76 of the accessory 70 engages the accessory receiving mechanism 50 of the pod 100 to removably secure the accessory 70 within the pod 100. The fluidic coupling of the accessory 70 is fluidically coupled/mated with the fluidic coupling 30 in this position, such that a managed flow of gas from the flow management device of the pod 100 can flow through the accessory 70 and out of the outlet 73, or a flow of gas can flow into the outlet 73 through accessory 70 and flow management device of the pod 100 to a source (e.g. suction operation). Tubing is configured to be connected to the outlet 73 for delivery to the patient worn device.

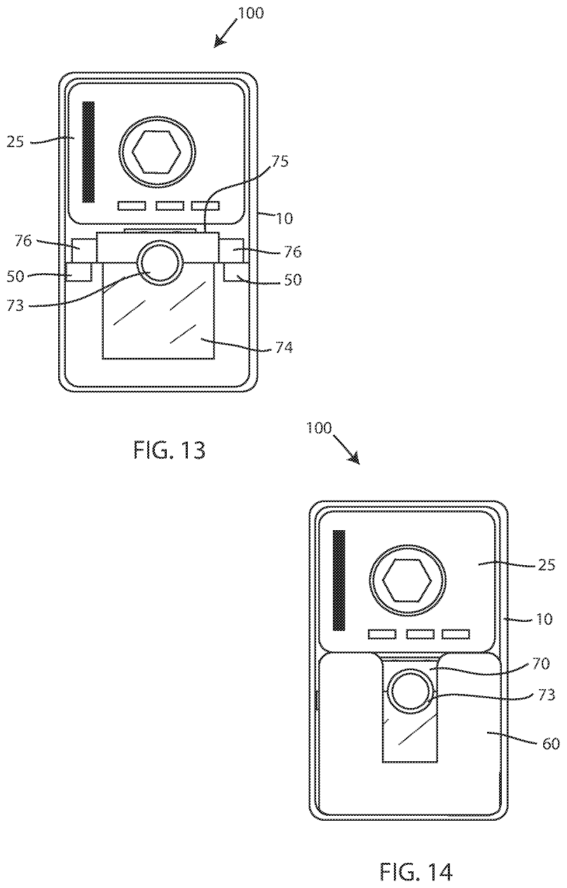

FIG. 14 depicts a schematic view of the pod 100 with an accessory inserted therein and a cover 60 covering the accessory 70, in accordance with embodiments of the present invention. In the illustrated embodiment, the pod 100 includes a cover 60 that is configured to cover at least a portion of the inserted accessory 70. The cover 60 is optionally used to prevent incidental contact with the accessory 70, deter non-clinicians from removing the accessory 70, hide contents of the accessory 70, etc. The cover 60 has a center opening for quick visual inspection of whether an accessory is inserted in the pod 100 or not. The cover 60 can be permanently attached to the casing 10 so that cover 60 (e.g. via a hinge connection) or the cover 60 can be non-permanently attached to the casing 10, configured to be snapped into place and detached from the casing 10.

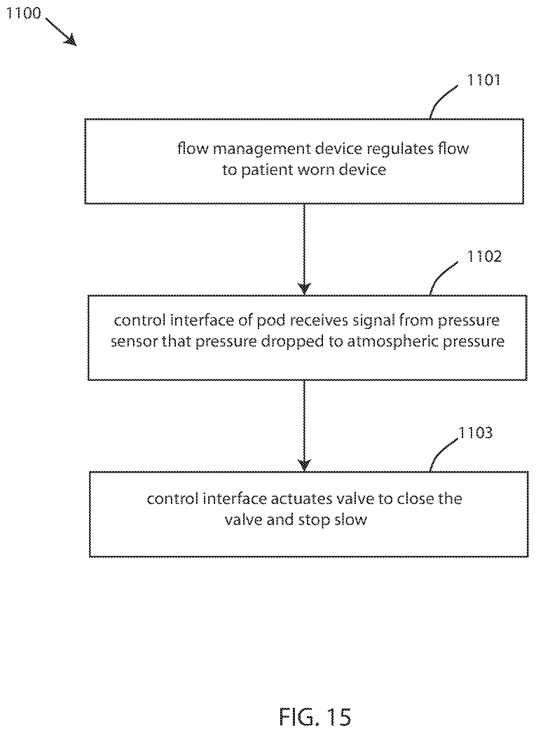

FIG. 15 depicts a flowchart of a method 1100 for automatic shutoff of gas supply to a patient worn device, in accordance with embodiments of the present invention. In step 1101, the flow management device 20 of the pod 100 regulates a flow of gas to a patient worn device at a pressure greater than atmospheric pressure. The pod 100 is configured to receive an accessory 70 having an outlet 73 and a pressure sensor 78 disposed proximate the outlet 73 of the accessory 70. To establish a fluidic connection between the flow management device 20 of the pod 100 and the patient worn device, a first end of a tubing is attached to the outlet 73 of the accessory 70 and a second, opposing end of the tubing is attached to a patient worn device. In step 1102, the control interface 25 of the pod receives a signal from the pressure sensor 78 of the accessory 70 indicating that the pressure has dropped to atmospheric pressure. The pressure drop to atmospheric pressure is caused by a disconnection of the fluidic connection between the flow management device 20 and the patient worn device. In an exemplary embodiment, the disconnection of the fluidic connection is a result of the outlet 73 of the accessory 70 being open to the atmosphere because the tubing is removed or otherwise dislodged from the outlet 73 of the accessory. In an alternative embodiment, the disconnection of the fluidic connection is a result of the second end of the tubing being open to the atmosphere because the patient worn device has been disconnected from the tubing. In step 1103, the control interface 25 of the pod 100 transmits a control signal to the flow management device 20 to stop the flow of gas to the patient worn device. For instance, the control interface 25 actuates a valve to stop the flow of gas. The valve may be an electronic solenoid valve, needle valve, etc., which can be electromechanically opened and closed via electronic control signals sent from the control interface 25.

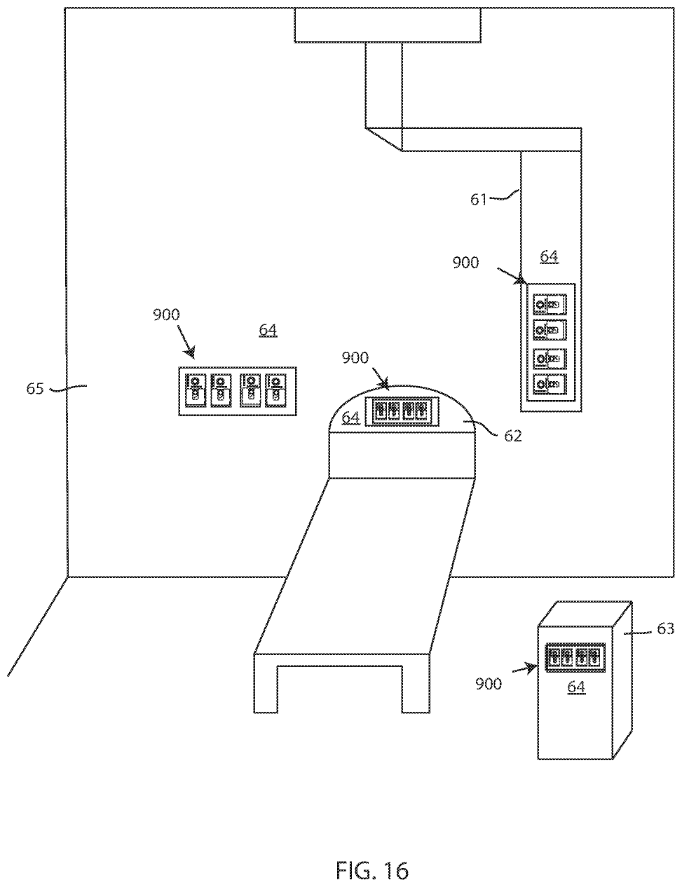

FIG. 16 shows a pod system 900 incorporated into various objects in an environment, according to embodiments of the present invention. The pod system 900 includes a plurality of pods 100 having flow management devices 20 that are at least partially recessed within finished surface 64, capable of receiving accessories with pressure sensors. The finished surface 64 can be a wall 65 of a room, a headwall 62 of a bed unit, an exterior surface of a freestanding gas delivery system 63, a ceiling column, a boom, and/or an exterior surface of a boom 61 of a gas delivery system. Fluidic connections between each pod 100 and the rough-in fluidic connections of the gas sources are located behind finished surface 64. Because the pod system 900 is recessed within the finished surface 64, the SOT devices do not protrude from the finished surface 64 and accessibility is limited. As a result, the pod system 900, and the pods therein, are largely protected from damage from incidental contact, clinicians and non-clinicians cannot easily remove the SOT devices from the wall in a hospital room to use in another room, and the fluidic connections to the gas sources are protected and located behind the finished surface 64 so that accidental disconnection leading to unintentional gas flow into the room cannot happen. The pod system 900 includes a receptacle recessed within finished surface 64, such as rough-in assembly 7, or a receptacle designed as several rough-in assemblies as one unit. Moreover, the pod system 900 includes the removable accessories configured to be fluidically coupled to the plurality of pods. Fluidic connections between each removable accessory and each pod are located within the pod and behind finished surface 64. A power source is electrically coupled to a control interface of each pod of the pod system 900 for controlling a flow through the pods 100; the power source may include one or more electrical wires running from a remote breaker behind the finished surface 64.

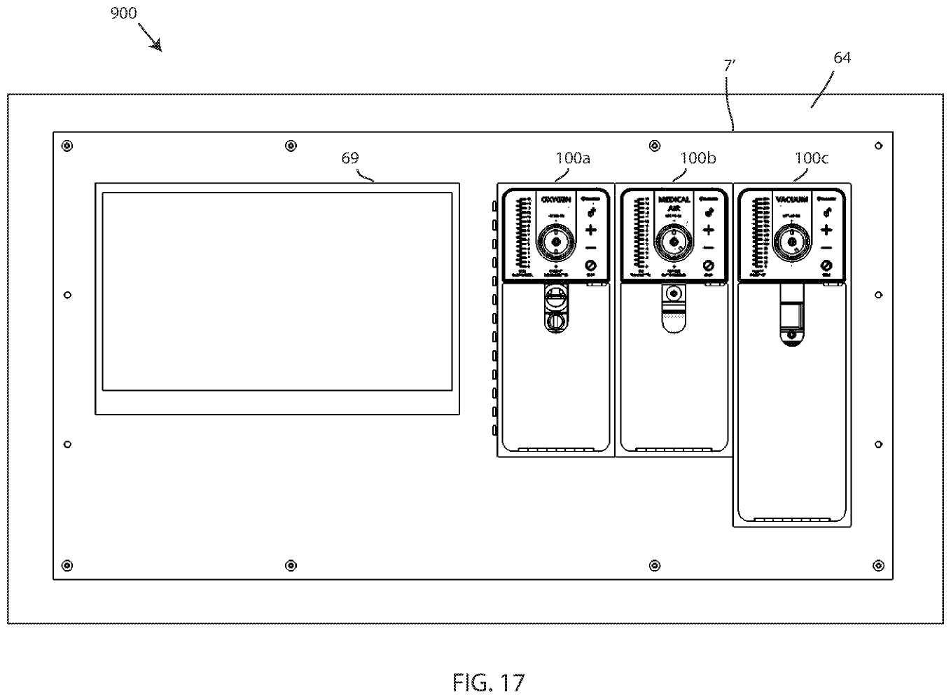

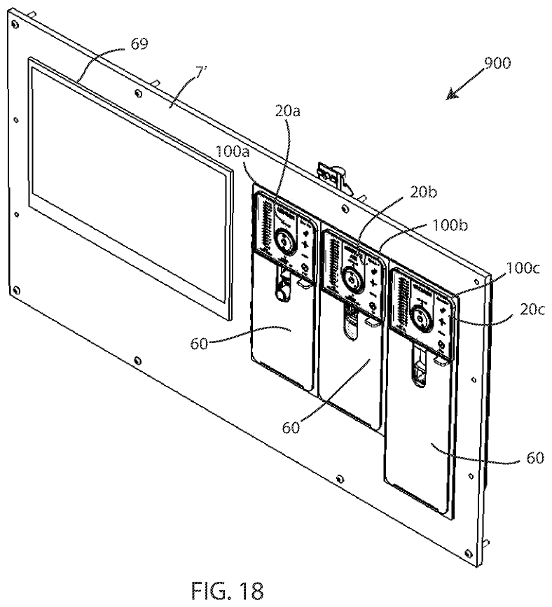

FIGS. 17-18 depict a pod system 900 according to an exemplary embodiment of the present invention. The pod system 900 illustrated in FIG. 17 includes a rough-in assembly cover 7′ designed with openings to accommodate three pods 100a, 100b, 100c and a display 69 for displaying various patient-related information, room information, pod and pod system information, location information, etc. The display 69 is also used to communicate notifications generated by the control interface 25 to indicate that the flow of gas has been shutoff, and an action(s) needs to be taken. The rough-in assembly cover 7′ is installed onto the finished surface 64 and cooperates with/is attached to rough-in assembly 7 installed behind the finished surface 64 that supports the fluidic connections to the sources 7′. The rough-in assembly cover 7′ may be attached to rough-in assembly 7 behind the wall prior to or after the pods 100a, 100b, 100c are removably inserted into the rough-in assembly. As described above, each pod is specific to a type of gas associated with a source. For example, pod 100a is specifically designed for managing a flow of gas associated with a first source, pod 100b is specifically designed for managing a flow of gas associated with a second source, and pod 100c is specifically designed for managing a flow of gas associated with a third source. Each pod 100a. 100b, 100c is removably inserted such that each pod 100a, 100b, 100c is substantially recessed within or otherwise located behind the finished surface 64.

The flow management devices of the pods 100a, 100b, 100c and accessories installed within the pods 100a, 100b, 100c are located behind the finished surface 64. A depth of the casings 10a, 10b, 10c can be sized and dimensioned to accommodate the flow management devices 20 of the pods 100a, 100b, 100c, and the accessories 70a, 70b, 70c removably inserted into the pod 100a, 100b, 100c, respectively. The casings 10a, 10b, 10c extend into the rough-in assembly 7 installed behind the finished surface 64, which allows for the flow management devices 20 and the accessories 70a, 70b, 70c to be supported behind the finished surface 64 of a hospital room wall, for example. FIG. 18 is a perspective view of pod system 900 shown in FIG. 17, with a cover 60 installed onto the pods, in accordance with embodiments of the present invention. The cover 60 is optionally removably installed over the accessories to prevent incidental contact with the accessory 70, deter non-clinicians from removing the accessory 70, hide contents of the accessory 70, etc.

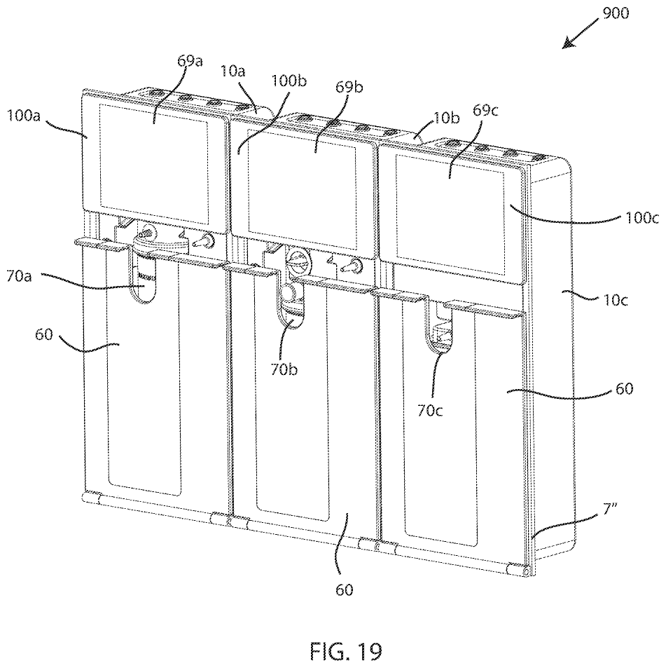

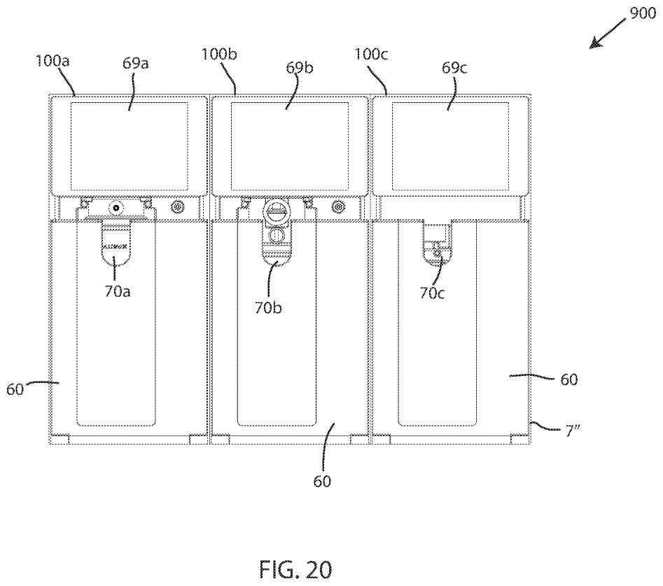

FIGS. 19-20 depict another embodiment of a pod system 900. The pod system 900 illustrated in FIG. 19 includes a rough-in assembly cover 7″ designed with openings to accommodate three pods 100a, 100b, 100c and, unlike the embodiment shown in FIGS. 17-18, a display 69a, 69b, 69c disposed on a front side of the pod 100a, 100b, 100c instead of a single display 69 to the side of the pods, for displaying various patient-related information, room information, pod and pod system information, location information, etc. The displays 69a, 69b, 69c can be used to display notifications triggered by the pressure sensor 78. The rough-in assembly cover 7″ is installed onto the finished surface and cooperates with/is attached to rough-in assembly 7 installed behind the finished surface 64 that supports the fluidic connections to the sources. The rough-in assembly cover 7″ may be attached to rough-in assembly 7 behind the wall prior to or after the pods 100a, 100b, 100c are removably inserted into the rough-in assembly 7. As described above, each pod is specific to a type of gas associated with a source; more than one pod may contain the same type of gas, or each pod is associated with a different type of gas. For example, pod 100a is specifically designed for managing a flow of gas associated with a first source, pod 100b is specifically designed for managing a flow of gas associated with a second source, and pod 100c is specifically designed for managing a flow of gas associated with a third source. Each pod 100a. 100b, 100c is removably inserted such that each pod 100a, 100b, 100c is substantially recessed within or otherwise located behind the finished surface.

As shown in FIGS. 19 and 20, the flow management devices of the pods 100a, 100b, 100c and accessories installed within the pods 100a, 100b, 100c are located behind the finished surface when the rough-in assembly cover 7″ is placed over the rough-in assembly 7 located behind the wall. FIG. 19 is a perspective view of pod system 900 and FIG. 20 is a front view of the pod system 900, in accordance with embodiments of the present invention. A depth of the casings 10a, 10b, 10c of the pods 100a, 100b, 100c is depicted in FIG. 19. The depth of the casings 10a, 10b, 10c can be sized and dimensioned to accommodate the flow management devices 20 of the pods 100a, 100b, 100c, and the accessories 70a, 70b, 70c removably inserted into the pod 100a, 100b, 100c, respectively. The casings 10a, 10b, 10c extend into the rough-in assembly 7 installed behind the finished surface, which allows for the flow management devices 20 and the accessories 70a, 70b, 70c to be supported behind the finished surface of a hospital room wall, for example. A cover 60 is installed onto the pods, which is optionally removably installed over the accessories to prevent incidental contact with the accessory 70, deter non-clinicians from removing the accessory 70, hide contents of the accessory 70, etc.

The pods 100a, 100b, 100c include a flow management device and at least one fluidic coupling within the pod casing 10a, 10b, 10c that is fluidically connected to the flow management device, as described above. The casings include one or more knockouts to facilitate fluidic connections with fluid sources located remotely from the pods 100a, 100b, 100c. The at least one fluidic coupling is configured to fluidically connect with a removable accessory 70a, 70b, 70c insertable within the pod casing 10a, 10b, 10c.

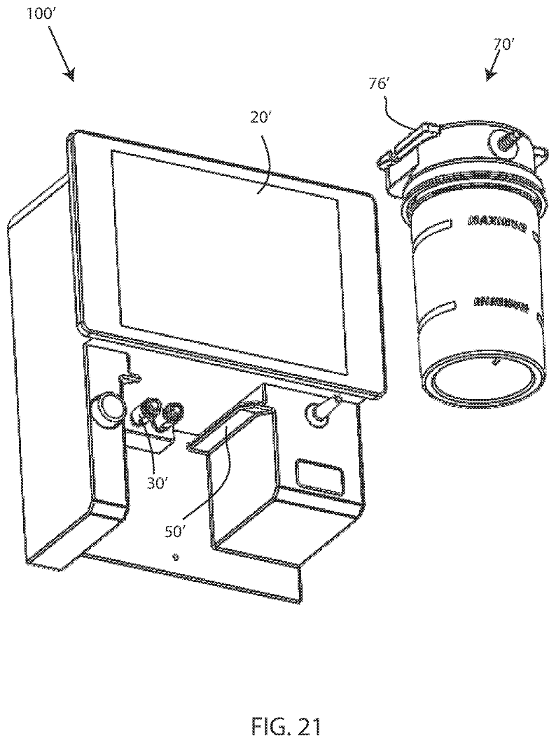

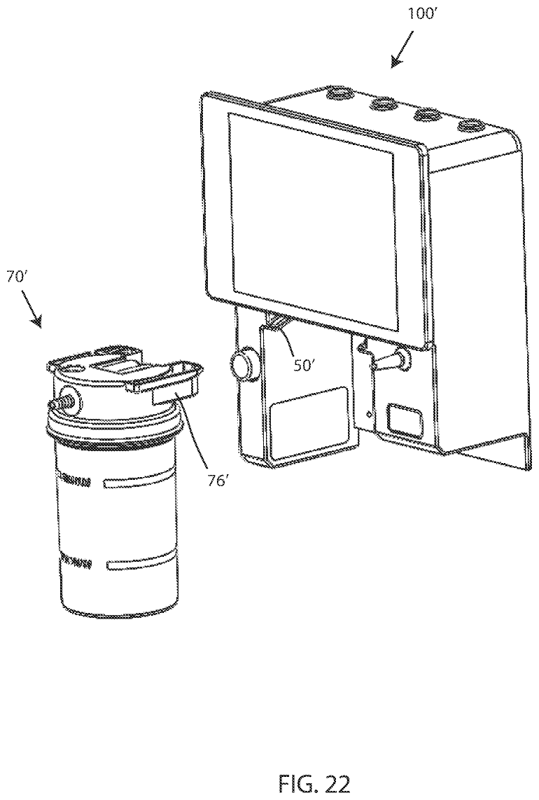

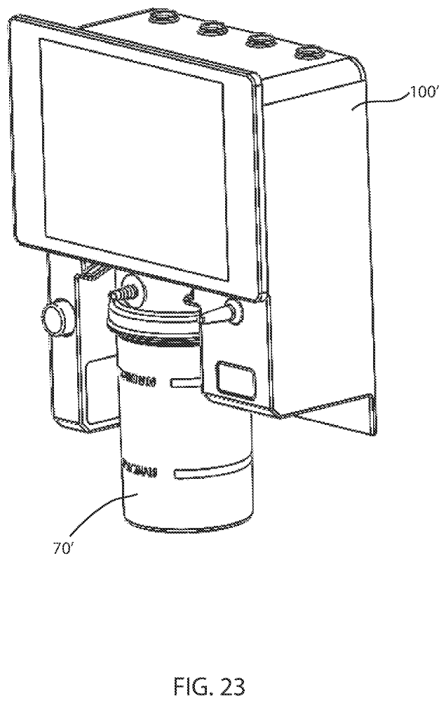

FIGS. 21-23 depict a connection between an accessory 70′ and a pod 100′, in accordance with an exemplary embodiment of the present invention. The accessory 70′ is configured to fluidically and mechanically connect to the fluidic coupling 30′ built into the pod 100′ so that the gas flows through the accessory 70′ and out to the patient worn device. One accessory is insertable into the pod 100′ at a given time, but there are many different accessories that can be used with pod 100′. The accessory 70′ resides within the pod casing (not shown in FIGS. 21-23) when inserted or otherwise attached to the pod 100′, and is removable from the pod 100′ so that a new accessory can be inserted into the pod 100′. Underneath the flow management device 20′ resides accessory receiving mechanism 50′. The accessory receiving mechanism 50′ facilitates an insertion and fluidic coupling of the accessory 70′. The accessory receiving mechanism 50′ includes a first receiving structure 51′ and a second receiving structure 52′ on a second side opposite the first side. The first receiving structure 51′ and the second receiving structure 52′ extend towards the rear wall of the casing, starting proximate or at the front of the casing. In the illustrated embodiment shown in FIGS. 21-23, the accessory receiving mechanism 50′ includes a ramped surface proximate the front side of the pod 100 that initially makes contact with and helps guides the pod engagement mechanism 76′ of accessory 70′ into the casing and into proper mating position with the fluidic coupling 30′ of the pod 100′. The ramped surface flattens out to support, at least partially, the weight of the accessory 70′ when inserted and mated within the pod 100′. The accessory 70′ can be driven up the ramped surface and towards the back of the pod 100′ by a user, guided by the mechanical interaction between the accessory receiving mechanism 50′ and the pod engagement mechanism 76′. With continued driving force, the accessory 70′ moves to a fully inserted position and mates with the fluidic coupling 30′ as a function of the driving force applied the accessory 70′. FIG. 22 depicts the accessory 70′ and the pod 100′ prior to being inserted into the pod 100′ and FIG. 23 depicts the accessory 70′ fully inserted within the pod 100′ such that the accessory 70′ is in fluidic communication with the flow management device 20′ by way of the fluidic coupling 30′.

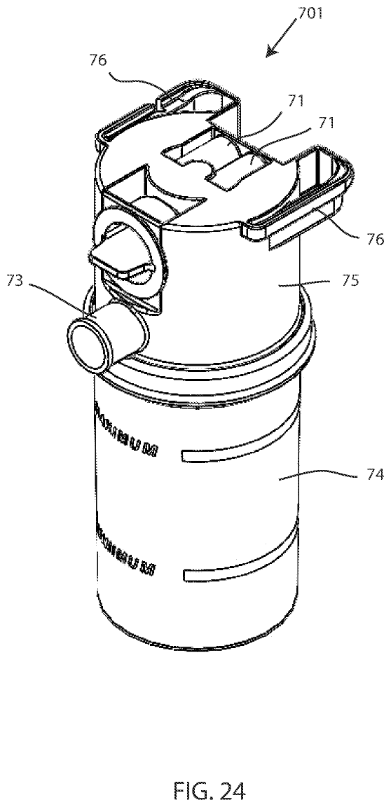

As described above, embodiments of the pod system 900 are compatible with many different types of removable accessories that are each capable of being inserted into a pod. FIGS. 24-26 depict example accessories that can be used with the pod system 100. FIG. 23 depicts accessory 701 which is a nebulizer used in conjunction with the flow of oxygen managed by a pod of pod system 100. The removable accessory 701 includes a pressure sensor integrated into the body portion 75 that is a lid or cover having a coupling 71 and an outlet 73. The accessory 701 includes a pod engagement mechanism 76 disposed on the body portion 75 for proper insertion and connection to a pod. The pod engagement mechanism 76 includes a first engagement structure at a first location on the body portion 75 and a second engagement structure at a second location of the body portion, opposite the first location. The first engagement structure and the second engagement structure of the pod engagement mechanism 76 is a protrusion or other lip or extension of the body portion 75 that is configured to cooperate with an accessory receiving mechanism of the pod. The accessory 701 includes incudes a canister 74 removably attached (e.g. threaded) to the body portion which functions as a container or storage device that has an interior space that allows a flow of oxygen (or other gas) between the fluidic coupling 71 that mates with the fluidic coupling of the pod and the outlet 73. The interior space of the canister 74 may store a fluid, such as water, for humidifying the oxygen flowing through the accessory 701.

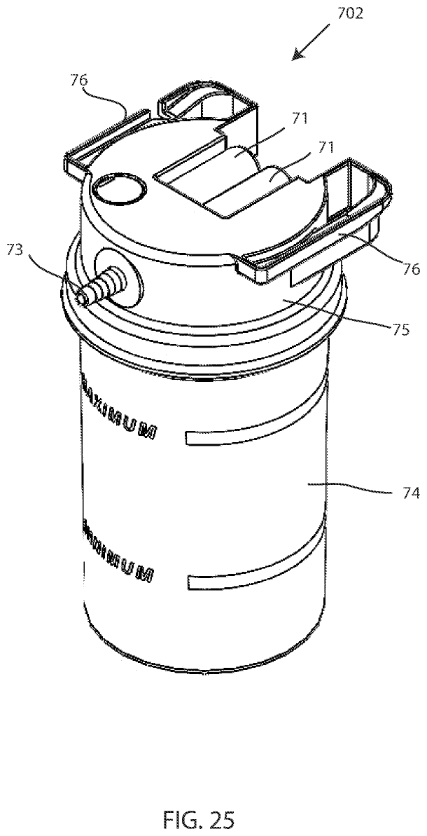

FIG. 25 depicts accessory 702 which is a humidifier used in conjunction with the flow of medical air managed by a pod of pod system 100. The removable accessory 702 includes a pressure sensor integrated with a body portion 75 that is a lid or cover having a coupling 71 and an outlet 73. The accessory 702 includes a pod engagement mechanism 76 disposed on the body portion 75 for proper insertion and connection to a pod. The pod engagement mechanism 76 includes a first engagement structure at a first location on the body portion 75 and a second engagement structure at a second location of the body portion, opposite the first location. The first engagement structure and the second engagement structure of the pod engagement mechanism 76 is a protrusion or other lip or extension of the body portion 75 that is configured to cooperate with an accessory receiving mechanism of the pod. The accessory 702 includes incudes a canister 74 removably attached (e.g. threaded) to the body portion which functions as a container or storage device that has an interior space that allows a flow of medical air (or other gas) between the fluidic coupling 71 that mates with the fluidic coupling of the pod and the outlet 73. The interior space of the canister 74 may store a fluid, such as water, for humidifying the medical air flowing through the accessory 701.

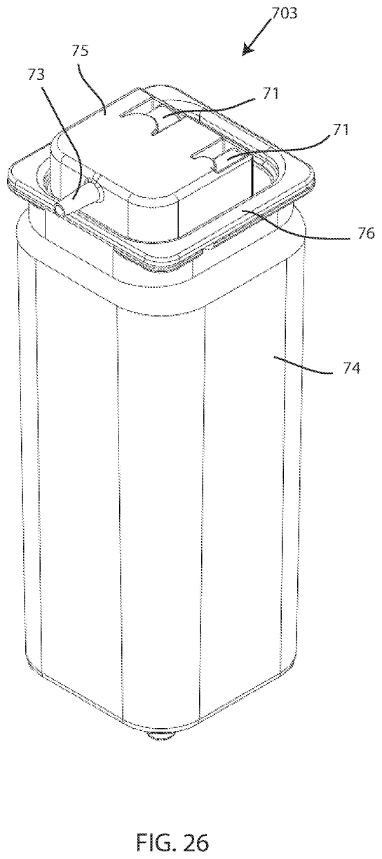

FIG. 26 depicts accessory 703 which is a vacuum container used in conjunction with a vacuum managed by a pod of pod system 100. The removable accessory 703 includes a pressure sensor integrated with a body portion 75 that is a lid or cover having a coupling 71 and an outlet 73. The accessory 703 includes a pod engagement mechanism 76 disposed on the body portion 75 for proper insertion and connection to a pod. The pod engagement mechanism 76 is a protrusion or other lip or extension of the body portion 75 that is configured to cooperate with an accessory receiving mechanism of the pod. The accessory 703 includes incudes a canister 74 removably attached (e.g. threaded) to the body portion which functions as a container or storage device that has an interior space that allows for storage of fluid and biological material drawn into the accessory 703 via the suction force into the accessory 703.

While this disclosure has been described in conjunction with the specific embodiments outlined above, it is evident that many alternatives, modifications and variations will be apparent to those skilled in the art. Accordingly, the preferred embodiments of the present disclosure as set forth above are intended to be illustrative, not limiting. Various changes may be made without departing from the spirit and scope of the invention, as required by the following claims. The claims provide the scope of the coverage of the invention and should not be limited to the specific examples provided herein.

What is claimed is:

1. A method comprising:
controlling a flow of gas to a patient worn device at a pressure greater or less than atmospheric pressure;
detecting a pressure drop to atmospheric pressure; and
in response to the detecting, automatically discontinuing the flow of the gas to the patient worn device.

2. The method of claim 1, wherein a flow management device of a suction and oxygen therapy device controls the flow to the patient worn device, and a fluidic connection is established by tubing connected at a first end to the flow management device and at a second end to the patient worn device.

3. The method of claim 2, wherein the pressure drop is caused by a disconnection of the fluidic connection between the flow management device and the patient worn device.

4. The method of claim 3, wherein the disconnection is a result of the first end of the tubing being removed from an outlet of the flow management device.

5. The method of claim 1, wherein the automatically discontinuing of the flow of the gas to the patient includes closing a valve.

6. The method of claim 3, wherein the disconnection is a result of the second end of the tubing being removed from the patient worn device.

7. A method comprising:
regulating, by a flow management device of a pod, a flow of gas to a patient worn device at a pressure greater or less than atmospheric pressure, the pod configured to receive an accessory having an outlet and a pressure sensor disposed proximate the outlet of the accessory;
receiving, by a control interface of the pod, a signal from the pressure sensor of the accessory indicating that the pressure has dropped to atmospheric pressure; and
transmitting, by the control interface of the pod, a control signal to the flow management device to stop the flow of gas to the patient worn device.

8. The method of claim 7, wherein a valve of the flow management device is closed as a function of the control signal.

9. The method of claim 7, wherein the pressure drop is caused by a disconnection of a fluidic connection between the flow management device and the patient worn device.

10. The method of claim 9, wherein the fluidic connection is established by tubing connected at a first end to the outlet of the accessory and at a second end to the patient worn device.

11. The method of claim 9, wherein the disconnection is a result of a first end of a tubing being removed from the outlet of the accessory.

12. A removable accessory for use with a pod, the removable accessory comprising:
a body portion having a first side and a second side;
at least one fluidic coupling disposed on the body portion configured to be fluidically coupled to a flow management device of the pod, as a function of the removable accessory being inserted within a pod casing of the pod;
an outlet fluidically connected to the at least one fluidic coupling, disposed on the body portion and configured to be attached to a tubing that fluidically connects the removable accessory to a patient worn device; and
a sensor disposed proximate the outlet and configured to detect a pressure differential within the body portion of the accessory.

13. The removable accessory of claim 12, wherein the sensor transmits a signal to the pod to discontinue a flow of gas to the patient worn device if the pressure differential indicates a pressure proximate the outlet is at or near atmospheric pressure.

14. The removable accessory of claim 12, wherein the sensor is integrated within the body portion.

15. The removable accessory of claim 12, wherein the sensor is in fluidic communication with the tubing attached to the outlet.

16. The removable accessory of claim 12, further comprising a canister operably attached to the body portion, wherein the canister contains a liquid for humidifying a fluid passing through the removable accessory.

17. A system comprising:
a pod comprising a flow management device disposed within a pod casing, at least one fluidic coupling within the casing that is fluidically connected to the flow management device;
a removable accessory for use with the pod, the removable accessory comprising: a body portion having a first side and a second side, at least one fluidic coupling disposed on the body portion that fluidically connects with the at least one fluidic coupling within the casing of the pod as a function of the removable accessory being inserted into the pod, and an outlet;
a sensor configured to detect a change in pressure at the outlet of the removable accessory; and
a control interface electrically connected to the sensor and configured to receive a signal from the sensor indicating the change in pressure, wherein the control interface sends a control signal to the flow management device to stop a gas from flowing through the outlet to a patient worn device.

18. The system of claim 17, further comprising a tubing connected at a first end to the outlet of the removable accessory and at a second end to the patient worn device, so that the gas delivered to the patient worn device flows from the outlet to the patient worn device when the tubing is connected at both the first end and the second end of the tubing.

19. The system of claim 17, wherein the change in pressure reflects a pressure drop to atmospheric pressure at the outlet as a result of the patient worn device being fluidically disconnected from the outlet.

20. The system of claim 17, wherein the change in pressure reflects a pressure drop to atmospheric pressure at the outlet as a result of the tubing being removed from the outlet.

21. A pod comprising:
a flow management device disposed within a pod casing, the flow management device electrically coupled to a control interface;
at least one fluidic coupling within the casing that is fluidically connected to the flow management device, the at least one fluidic coupling configured to fluidically connect with a removable accessory insertable within the pod casing; and
a receptacle within the casing that is configured mate with a pressure sensor of the removable accessory, the receptacle electrically coupled to the control interface so that when the removable accessory is inserted within the pod casing, data from the pressure sensor is received by the control interface.
